(19) United States
(12) Patent Application Publication
(10) Pub. No.: US 2002/0168633 A1
(43) Pub. Date: Nov. 14, 2002

(54) NUCLEOTIDE FRAGMENT OF THE 23S RNA OF THE GENUS CHLAMYDIA, USE AS A PROBE, PRIMER, AND IN A REAGENT AND A DETECTION PROCEDURE

(76) Inventors: CLAUDE MABILAT, SAINT GERMAIN AU MONT D'OR (FR); KARL-HEINZ SCHLEIFER, UNTERSCHLEISSHEIM (DE); WOLFGANG LUDWIG, SACHSENKAM (DE)

Correspondence Address: OLIFF & BERRIDGE, PO BOX 19928, ALEXANDRIA, VA, 22320

(21) Appl. No.: 09/230,926

(22) PCT Filed: Jun. 5, 1998
(86) PCT No.: PCT/FR98/01157

(30) Foreign Application Priority Data
Jun. 5, 1997 (FR) 97 07200

(51) Int. Cl. C12Q 1/68; C07H 21/04

(52) U.S. Cl. 435/6; 536/23.7

(57) ABSTRACT

The invention concerns a single-stranded nucleotide fragment comprising at least a sequence of 12 contiguous nucleotide patterns, belonging to a 23S ribosome RNA strand of the species of the Chlamydia genus or to is complementary strand, said sequence being selected in any one of the following groups consisting of said RNA zones: Group I: 451-472, 542-570, 596-623, 731-756, 878-890, 996-1020, 1061-1094, 1123-1186, 1857-1880, 2234-2307, 2341-2370; Group II: 420-450, 473-514, 694-713, 756-790, 842-857, 927-937, 1231-1248, 1241-1319, 1371-1381, 1880-1895, 1943-1961, 2151-2182; Group III: 404-426, 436-457, 466-515, 683-722, 747-808, 817-863, 891-955, 1024-1055, 1208-1251, 1315-1350, 1407-1548, 1364-1388, 1576-1622, 1891-1918, 2148-218; the first number corresponding to the position of the first nucleotide of said zone relative to the 23S ribosome RNA nucleotide sequence of serotype A of Chlamydia trachomatis, SEQ ID NO:55, selected as reference sequence, and the second number corresponding to the position of the last nucleotide of said zone relative to this same reference sequence. The invention also concerns the uses of said fragment as probe, primer and in a reagent and method for detecting said bacteria.

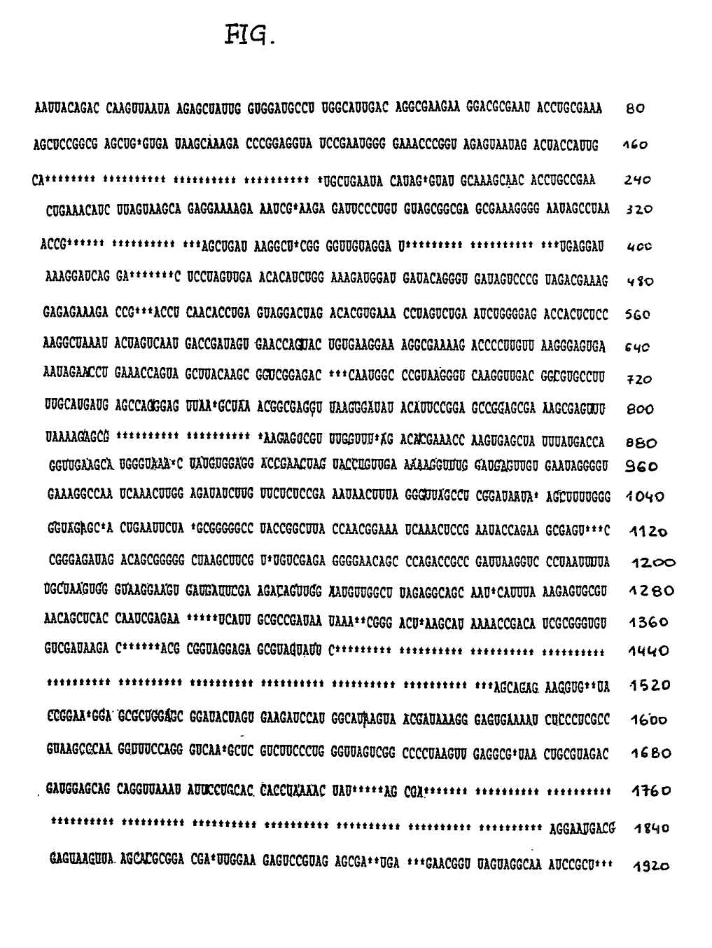

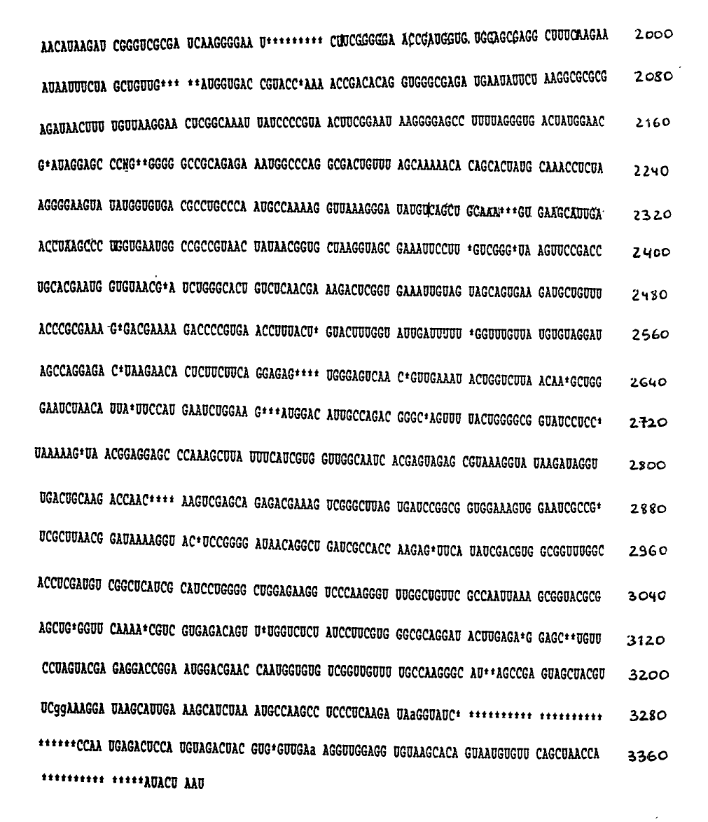

The present invention concerns the field of detection and/or amplification techniques, with the aid of oligonucleotide probes or primers, and their application in the search for the presence or for the identification of bacteria of the genus Chlamydia.

Three species of Chlamydia are presently known: Chlamydia trachomatis (subgroup A) which is specific to man and includes 15 serotypes, Chlamydia psittaci (subgroup B) which is commensal in birds and Chlamydia pneumoniae (Twar strain). The pathogenic power of these bacteria is very varied, just as much in man as in animals.

In man, Chlamydia trachomatis is especially responsible for urethritis, cervicitis or salpingitis, conjunctivitis, trachoma which can lead to blindness, arthritis, perihepatitis; two other clinical forms are also frequent, namely conjunctivitis of the newborn, who are infected in the course of delivery and the conjunctivitis known as swimming pool conjunctivitis, contracted in the course of bathing in a swimming pool whose water is dirty; Chlamydia psittaci contaminates man by the intermediary of bird excrement, causing attacks such as benign pneumopathy (ornithosis) or more serious pneumopathy (psittacosis) and pseudoinfluenza; finally, Chlamydia pneumoniae was isolated in 1965 from a conjunctival sample during a vaccine campaign against trachoma and in 1983 from a pharyngeal sample during an epidemic of acute respiratory illnesses in the United States. Recently, the studies of F. Blasi et al. (Journal of Clinical Microbiology, (November 1996) 2766-2769) demonstrated the direct implication of C. pneumonia [sic] in the etiology of atherosclerosis.

For a long time, the diagnosis of infections due to Chlamydia was based:

on the direct examination of the smears produced from samples obtained by conjunctival, urethral, cervical or anorectal scraping; however, this technique is tricky because it is necessary that the cells are correctly spread out and separated one from another to allow, after staining, the intracellular inclusions characteristic of Chlamydia infections to be demonstrated;

on the isolation of the bacterium by culture; however, the Chlamydia are particularly fragile bacteria, obligatory intracellular parasites which only multiply on live media such as fertilized eggs or cell cultures (McCoy or Hela 299 cells); for numerous years, only inoculation of the fertilized egg was practiced by specialized laboratories, and although cell cultures are to be used henceforth, this technique remains difficult to carry out and is always restricted to certain laboratories;

on the immunological diagnosis based either on immunoenzymatic (ELISA) or immunofluorescence (IF) techniques, or on complement immobilization reactions (CIR).

However, these different techniques did not allow a rapid and reliable diagnosis outside an obvious epidemiological context. It was thus important to develop a bacteriological diagnostic test sufficiently specific and sensitive to allow rapid and selective detection, in a sample, of bacteria belonging to the genus Chlamydia, and especially of Chlamydia trachomatis.

A first generation of rapid tests was proposed, based on the techniques of in situ hybridization (DUTIHL et al., Ann. Microbiol. 1988, 139, 115-118). However, this test using the total chromosomal DNA of Chlamydia trachomatis labeled as an oligonucleotide probe lacks specificity. This is because this DNA contains a large number of common sequences with the genome of other bacteria which can lead to false-positive detection tests. In addition, the preparation of such samples is tricky especially because of their method of preparation and their considerable size.

Other Chlamydia detection tests were developed based on nucleic acid amplification and detection tests (for example the test marketed by the company Hoffman la Roche based on the detection of a portion of the cryptic gene peculiar to the strain Chlamydia trachomatis or the test marketed by the company Abbott based on the demonstration of the gene coding for the major outer membrane protein of Chlamydia called MOMP).

The bacterial ribosomes contain at least three distinct ribosomal RNA molecules called 5S, 16S and 23S RNA. According to the procedure demonstrated in the present invention, the ribosomal RNA of the bacteria can be used as a target.

The use of the 16S RNA of Chlamydia in the diagnosis of infections due to these bacteria was described in 1990 by the company Genprobe which developed a chemiluminescence detection kit (PACE 2-assay).

Nucleotide probes having the 23S RNA of Chlamydiae for a target have now been discovered, which allow at least one group of species of the genus Chlamydia to be discriminated from other genera or groups of bacterial genera.

Before explaining the invention in more detail, different terms used in the description and the claims are defined below:

“nucleic acid extracted from bacteria” is understood as meaning either the total nucleic acid, or the ribosomal RNA, in particular the 23S rRNA. or the genomic DNA, or even the DNA obtained from the reverse transcription of the 23S ribosomal RNA;

a “nucleotide fragment”, or an “oligonucleotide” are two synonymous terms indicating a linkage of nucleotide groups characterized by the informational sequence of the natural nucleic (or possibly modified) acids and capable of hybridizing, like natural nucleic acids, with a complementary or approximately complementary nucleotide fragment, under predetermined conditions; the linkage can contain nucleotide groups of different structure to that of the natural nucleic acids; a nucleotide (or oligonucleotide) fragment can contain, for example, up to 100 nucleotide units; it generally contains at least 12 nucleotide units and can be obtained from a natural nucleic acid molecule and/or by genetic recombination and/or by chemical synthesis;

a nucleotide unit is derived from a monomer which can be a natural nucleic acid nucleotide whose constitutive elements are a sugar, a phosphate group and a nitrogenous base; in the DNA the sugar is 2-deoxyribose, in the RNA the sugar is ribose; depending on whether DNA or RNA is concerned, the nitrogenous base is chosen from adenine, guanine, uracil, cytosine, thymine; or else the monomer is a nucleotide modified in at least one of the three constitutive elements; by way of example, the modification can occur either at the level of the bases, with modified bases such as inosine, 5-methyldeoxycytidine, deoxyuridine, 5-dimethylaminodeoxyuridine, 2,6-diaminopurine, 5-bromodeoxyuridine or any other modified base capable of hybridization, or at the level of the sugar, for example the replacement of at least one deoxyribose by a polyamide (P. E. Nielsen et al., Science, 254, 1497-1500 (1991)), or even at the level of the phosphate group, for example its replacement by esters chosen especially from the diphosphates, alkyl- and arylphosphonates and phosphorothioates;

“informational sequence” is understood as meaning any ordered sequence of nucleotide-type units, whose chemical nature and order in reference terms constitute an item of information analogous to that given by the sequence of natural nucleic acids;

“hybridization” is understood as meaning the process in the course of which, under appropriate conditions, two nucleotide fragments having sufficiently complementary sequences are capable of associating by stable and specific hydrogen bonds to form a double strand; the hybridization conditions are determined by the “stringency”, that is to say the strictness of the operating conditions; the hybridization is all the more specific when it is carried out with greater stringency; the stringency is a function especially of the base composition of a probe/target duplex, as well as by the degree of mismatching between two nucleic acids; the stringency can likewise be a function of parameters of the hybridization reaction, such as the concentration and the type of ionic species present in the hybridization solution, the nature and the concentration of denaturing agents and/or the hybridization temperature; the stringency of the conditions under which a hybridization reaction must be carried out depends especially on the probes used; all these data are well known and the appropriate conditions can possibly be determined in each case by routine experiments; in general, depending on the length of the probes used, the temperature for the hybridization reaction is between approximately 20 and 65° C., in particular between 35 and 65° C. in a saline solution at a concentration of approximately 0.3 to 1M; in particular, under the hybridization conditions according to the present invention, a temperature of 37° C.±1° C. is chosen, in a 3×PBS saline solution (NaCl 0.45 m; sodium phosphate 0.15 M)

a “probe” is a nucleotide fragment comprising, for example, 12 to 100 nucleotide units, especially 12 to 35 nucleotide units, possessing a hybridization specificity under conditions determined so as to form a hybridization complex with a target nucleic acid having, in the present case, a nucleotide sequence comprised either in a ribosomal RNA, or in a DNA obtained by reverse transcription of said ribosomal RNA, or alternatively in a DNA (called ribosomal DNA or rDNA here) of which said ribosomal RNA is the product of transcription; a probe can be used for diagnostic purposes (especially capture or detection probes) or for purposes of therapy;

a “capture probe” is immobilized or immobilizable on a solid support by any appropriate means, for example by covalence, by adsorption, or by direct synthesis on a solid support (see especially patent application WO 92/10092);

a “detection probe” can be labeled by means of a label chosen, for example, from the radioactive isotopes, enzymes, in particular enzymes capable of acting on a chromogenic, fluorigenic or luminescent substrate (especially a peroxidase or an alkaline phosphatase), chromophoric chemical compounds, chromogenic, fluorigenic or luminescent compounds, analogs of nucleotide bases, and ligands such as biotin;

a probe of the invention can also be used for therapeutic purposes, as an antisense probe capable of inhibiting protein synthesis by specifically blocking the translation of the messenger RNAs into proteins.

a “primer” is a probe comprising, for example, from 12 to 100 nucleotide units and possessing a hybridization specificity under determined conditions for the initiation of an enzymatic polymerization, for example in an amplification technique such as PCR (Polymerase Chain Reaction), in a sequencing procedure, in a reverse transcription method, etc.

The probes according to the invention can be used, for diagnostic purposes, in investigating the presence or the absence of a target nucleic acid in a sample, according to all the known hybridization techniques and especially the techniques of point deposition on filter, called “DOT-BLOT” (MANIATIS et al., Molecular Cloning, Cold Spring Harbor, 1982), the DNA transfer techniques called “SOUTHERN BLOT” (SOUTHERN, E. M., J. Mol. Biol., 98, 503 (1975)), the RNA transfer techniques called “NORTHERN BLOT”, or the techniques called “sandwich” (DUNN A. R., HASSEL J. A., Cell, 12, 23 (1977)); use is made in particular of the sandwich technique, with a capture probe and/or a detection probe, said probes being capable of hybridizing with two different regions of the target nucleic acid, and at least one of said probes (generally the detection probe) being capable of hybridizing with a region of the target which is specific for the species or the group of species investigated, it being understood that the capture probe and the detection probe must have nucleotide sequences which are at least partly different.

“equivalent sequence” to a sequence typically described according to the present invention is understood as meaning a sequence which, under predetermined hybridization conditions, such as those defined previously, ensures the same specificity as the sequence described.

Under conditions which are specified in Example 1 below, the nucleotide sequence of the rDNA corresponding to the 23S ribosomal RNA of 12 serotypes of Chlamydia trachomatis and of one strain of C. psittaci and of C. pneumoniae was determined.

Thus, the invention provides:

(a) a single-stranded nucleotide fragment of the invention which comprises at least a sequence of 12 contiguous nucleotide units, belonging to a strand of the 23S ribosomal RNA of the species of the genus Chlamydia or to its complementary strand, this sequence being chosen from any one of the following groups formed by zones of said RNA:

Group I: 451-472, 542-570, 596-623, 731-756, 878-890, 996-1020, 1061-1094, 1123-1186, 1857-1880, 2234-2307, 2341-2370;

Group II: 420-450, 473-514, 694-713, 756-790, 842-857, 927-937, 1231-1248, 1241-1319, 1371-1381, 1880-1895, 1943-1961, 2151-2182;

Group III: 404-426, 436-457, 466-515, 683-722, 747-808, 817-863, 891-955, 1024-1055, 1208-1251, 1315-1350, 1407-1548, 1364-1388, 1576-1622, 1891-1918, 2148-218 [sic];

the first number corresponding to the position of the first nucleotide of said zone with respect to the nucleotide sequence of the 23S ribosomal RNA of the A serotype of Chlamydia trachomatis, SEQ ID NO: 55, chosen as a reference sequence and illustrated in the FIGURE, and the second number corresponding to the position of the last nucleotide of said zone with respect to this same reference sequence; preferably, a fragment of the invention comprises a sequence of at least 12 contiguous nucleotide units, included in a nucleotide sequence chosen from the sequences SEQ ID NO: 1 and SEQ ID NO: 3 to SEQ ID NO: 54, and their complementary sequences, or better still, said fragment consists of a nucleotide sequence chosen from the sequences SEQ ID NO: 1 and SEQ ID NO: 3 to SEQ ID NO: 54, and their complementary sequences;

(b) a single-stranded nucleotide fragment of DNA, obtained by reverse transcription of an above nucleotide fragment defined according to (a), or its complementary fragment;

(c) a single-stranded nucleotide fragment of genomic DNA, whose transcription product is an above nucleotide fragment defined according to (a), or its complementary fragment.

Other subjects of the invention are the following:

(d) a probe for the specific detection of bacteria of the genus Chlamydia, which comprises, or which advantageously consists of, a sequence of at least 12, preferably 18, or better still 20, contiguous nucleotide units, included in a sequence chosen from the sequences SEQ ID NO: 1 and SEQ ID NO: 3 to SEQ ID NO: 12, their complementary sequences and their equivalent sequences;

(e) a probe for the specific detection of bacteria of the species Chlamydia trachomatis, which comprises, or which advantageously consists of, a sequence of at least 12, preferably 18, or better still 20, contiguous nucleotide units included in a sequence chosen from the sequences SEQ ID NO: 13 to SEQ ID NO: 24, their complementary sequences and their equivalent sequences;

(f) a probe for the specific detection of bacteria of the species Chlamydia pneumoniae, which comprises, or which advantageously consists of, a sequence of at least 12, preferably 18, or better still 20, contiguous nucleotide units, included in a sequence chosen from the sequences SEQ ID NO: 25 to SEQ ID NO: 39, their complementary sequences and their equivalent sequences;

(g) a probe for the specific detection of bacteria of the species Chlamydia psittaci, which comprises, or which advantageously consists of, a sequence of at least 12, preferably 18, or better still 20, contiguous nucleotide units, included in a sequence chosen from the sequences SEQ ID NO: 40 to SEQ ID NO: 54, their complementary sequences and their equivalent sequences.

As stated previously, a probe of the invention can be labeled by a tracer or immobilized on a solid support.

(h) a therapy probe for the treatment of infections due to a determined species of Chlamydia, which comprises or which consists of a sequence of at least 12, preferably 18, or better still 20, contiguous nucleotide units, included in a nucleotide sequence chosen from the sequences SEQ ID NO: 1 and SEQ ID NO: 3 to SEQ ID NO: 54, their complementary sequences and their equivalent sequences.

Another subject of the invention is a primer for the specific reverse transcription of a 23S ribosomal RNA sequence of a bacterium of the genus Chlamydia, into a complementary DNA sequence or a primer especially for enzymatic amplification, such as by chain polymerization reaction, of the DNA sequence which is complementary to a 23S ribosomal RNA sequence of Chlamydia, said primer comprising a sequence of at least 12, preferably 18, or better still 20, contiguous nucleotide units, included in a nucleotide sequence chosen from the sequences SEQ ID NO: 1 and SEQ ID NO: 3 to SEQ ID NO: 54, their complementary sequences and their equivalent sequences.

The invention also relates to a reagent for detecting and/or identifying and/or quantifying at least one species of Chlamydia, comprising at least one probe of the invention, and in particular a capture probe and a detection probe, one and/or the other corresponding to the definition of a probe according to the invention.

The reagent can comprise a mixture of probes of the invention with the aim of detecting at least two species of Chlamydia.

Finally, the invention provides a process for detecting and/or identifying and/or quantifying at least one species of Chlamydia, in a biological sample capable of containing at least one nucleic acid of said species, namely the 23S ribosomal RNA, extracted from Chlamydiae, possibly denatured, or the genomic, extracted and denatured DNA of bacteria, comprising the steps consisting in contacting said sample with at least one probe of the invention, and in detecting the hybridization of said probe by the formation of a hybridization complex between the probe and said nucleic acid. Preferentially, before exposing the DNA to the probe of the invention, amplification of this DNA is carried out in the presence of an adapted enzymatic system and at least one amplification primer of the invention and possibly a eubacterial primer.

The invention and some of these applications are explained below with Examples 1 and 2.

The FIGURE represents the nucleotide sequence of the 23S ribosomal RNA of the A serotype of C. trachomatis, used as a reference sequence to identify the zones of the 23S rRNA of the other species of Chlamydiae. In this sequence, A denotes adenine, G guanine, C cytosine, U uracil, N any one of the four abovementioned bases, * represents the absence of nucleotide in the position indicated.

### EXAMPLE 1

### Determination of the Nucleotide Sequence of the 23S Ribosomal RNAs of Chlamydia

The nucleotide sequence of the 23S ribosomal RNA of the following 14 strains was determined.

C. trachomatis serotype A ATCC VR571B, batch 6W, HAR13/92-02

C. trachomatis serotype C ATCC VR572, batch 8, HAR-32/89-01

C. trachomatis serotype D ATCC VR 885, batch 8, VW-3/Cx/03-89

C. trachomatis serotype Ba ATCC VR-347, batch 6, Apache-2/90-04

C. trachomatis serotype E ATCC VR3488, Baur batch 8W/92-02

C. trachomatis serotype F ATCC VR346, K-Cal-3, batch 10W/92-02

C. trachomatis serotype G ATCC 878, batch 7, 6/4/86

C. trachomatis serotype H ATCC VR-879, batch 8W, UW-43/CX/92-12

C. trachomatis serotype I ATCC VR 880, UW-12/UR, batch 10W/92-08

C. trachomatis serotype K ATCC VR887, VW-32/CX, batch 13 W/92-02

C. trachomatis serotype LGV2 ATCC 434, batch 13W/92-10

C. trachomatis serotype LGV3 ATCC VR903, batch 6/90-03

C. pneumoniae TWAR

C. psittaci Borg

The sequence of Chlamydia trachomatis serotype A, used as a reference sequence for determining the zones of the constitutive rRNA of the fragment of the invention is identified by SEQ ID NO: 55 and is represented on the FIGURE.

The total nucleic acid of the strains was isolated by centrifugation in cesium chloride. PCR amplification products which cover 90% of the sequence were generated from ribosomal RNA with the aid of amplification primers of eubacterial specificity.

The amplification products obtained were sequenced directly by the chain termination method (Sanger et al. Proc. Natl. Acad. Sci. USA, 1977, 74: 5463-5467), by thermal cyclization using a thermostable Taq DNA polymerase (Perkin) and migration on a LICOR sequencer.

### EXAMPLE 2

### Use of a Probe of the Invention Directed Against the 23S Ribosomal RNA for the Identification of Chlamydia trachomatis

The specificity of the probe commencing at the nucleotide No. 9 and finishing at the nucleotide No. 25 of the SEQ ID NO: 15 to 26, for the species C. trachomatis was verified.

Its sequence on the complementary strand is the following: ACC CTT ACG GGC CAT TG.

A collection of strains of various bacteria representing a section of the bacterial phylogenic tree was tested by PCR amplification of a conserved portion of the 23S ribosomal DNA with the aid of eubacterial primers. Each amplification product was tested with the putative C. trachomatis probe and the results confirmed the specificity of this probe.

The different serotypes of C. trachomatis and the isolates of C. psittaci and of C. pneumoniae were cultured in a McCoy monolayer cell. These cultures were recovered and an aliquot of 10 μl was resuspended directly in the PCR amplification tube.

The other bacterial species were cultured according to good bacteriological practices (Manual of clinical microbiology, fifth edition, 1991, Balows et al. ASM Eds., Washington D.C., USA). A strain of each following species was selected: Mycobacterium bovis BCG, Staphylococcus aureus, Enterococcus faecium, Listeria monocytogenes, Rhodobacter capsulata, Bordetella pertussis, Escherichia coli, Haemophilus influenzae, Campylobacter jejuni, Leptospira interrogans, Borrelia burgdorferi. These strains were lyzed according to various techniques and an aliquot of the lyzate was resuspended in the PCR tube.

The amplification of a eubacterial fragment of 900 base pairs including the zone to be tested was carried out according to the methodology of the following reference: Sallen et al. (1996) Comparative analysis of 16S and 23S rRNA sequences of Listeria species, Int. J. Sys. Bact. 46: 669-674. The PCR amplification primers were identical: 1f. TCC GAA TGG GGA AAC CC and 10r: GA(C/T)(C/T)AG TGA GCT RTT AC.

The hybridization of the amplified ribosomal DNAs was carried out according to the nonradioactive and semiautomated detection procedure described in the international patent application WO-91/19812 in the name of the Applicant and whose contents are incorporated in the present description by way of reference. A capture probe (27 nucleotides in C. trachomatis whose 3′ end corresponds to the nucleotide No. 30 of the SEQ ID NO: 15 to 26 and of sequence TCA TCA TGC AAA AGG CAC GCC GUC AAC) and an oligonucleotide-alkaline phosphatase detection conjugate (corresponding to the probe defined at the start of Example 2) are used.

The operation was carried out in the VIDAS automated machine (registered trade mark—marketed by the company bioMérieux-VITEK). The reaction chamber is the SPR (trade name) (“Solid Phase Receptacle”) which is a conical support produced from a material sold under the name K resin (butadiene-styrene copolymer) and marketed by the company bioMérieux. The various reagents are placed in a strip with several cuvettes and the different steps take place in the SPR which is capable of aspirating and of delivering the reagents and which thus serves as a pipette. The sandwich hybridization reaction occurs on the internal wall of the cone as described below.

On the internal surface of the SPR is passively immobilized the capture oligonucleotide containing at its 5′ end the Aminolink 2 ligand (Applied Biosystems—ref. 400808) at a concentration of 1 ng/μl in a volume of 315 μl of a 4× solution of PBS (200 mM sodium phosphate pH 7.0, 600 mM NaCl). After one night at ambient temperature or two hours at 37° C., the cones are washed twice with a PBS Tween solution, then dried in vacuo. In cuvettes, the strip contains the reagents necessary for the denaturation and the detection, that is to say: sodium hydroxide, acetic acid, 200 μl of a 0.1 ng/μl solution of the oligonucleotide-alkaline phosphatase detection conjugate, 2 times 600 μl of PBS Tween wash solution and a substrate cuvette.

In the first well of the strip are deposited 10 μl of the PCR product to be tested. After denaturation and neutralization, the product is incubated for 30 minutes with the detection probe, the cone is washed twice with a PBS Tween solution. 250 μl of MUP substrate (4-methylumbelliferyl phosphate) in solution in a diethanolamine buffer are aspirated in the cone, then released into a reading cuvette. The apparatus measures the fluorescent signal expressed in RFU (relative fluorescence units) of the cuvette.

The results obtained indicate that the combination of probes used is specific for C. trachomatis. It does not show any cross-reactions with the nucleic acids, in particular the ribosomal DNA, of species representing the bacterial families (phylogenic section). It was checked that the ribosomal DNA targets of the other species were indeed available for hybridization by visualizing these amplification products on agarose gel.

1. Single-stranded nucleotide fragment comprising at least one sequence of 12 contiguous nucleotide units, belonging to a strand of the 23S ribosomal RNA of the species of the genus Chlamydia or to its complementary strand, this sequence being chosen from any one of the following groups formed by zones of said RNA:
Group I: 451-472, 542-570, 596-623, 731-756, 878-890, 996-1020, 1061-1094, 1123-1186, 1857-1880, 2234-2307, 2341-2370;
Group II: 420-450, 473-514, 694-713, 756-790, 842-857, 927-937, 1231-1248, 1241-1319, 1880-1895, 1943-1961, 2151-2182;
Group III: 404-426, 436-457, 466-515, 683-722, 747-808, 817-863, 891-955, 1024-1055, 1208-1251, 1315-1350, 1407-1548, 1364-1388, 1576-1622, 1891-1918, 2148-218 [sic];
the first number corresponding to the position of the first nucleotide of said zone with respect to the nucleotide sequence of the 23S ribosomal RNA of the A serotype of Chlamydia trachomatis, SEQ ID NO: 55, chosen as a reference sequence, and the second number corresponding to the position of the last nucleotide of said zone with respect to this same reference sequence, with the exception of the following sequences
GAGGUCGGUC UUUCUCUCCU UUCGUCUACG,
CCGGGGCUCC UAUCGUUCCA UAGUCACCCU AAAAG,
CGGUCUUUCU CUCCUUUCGU CUACGGG,
CGCUGGGUAA UCACCUUAAG,
CCGGGGCUCC UAUCGUUCCA UAGUCACCCU AAAAG.

2. Fragment according to claim 1, characterized in that it comprises a sequence of at least 12 contiguous nucleotide units, included in a nucleotide sequence chosen from the sequences SEQ ID NO: 1, SEQ ID NO: 3 to SEQ ID NO: 18, SEQ ID NO: 20, SEQ ID NO: 22 to SEQ ID NO: 54 and their complementary sequences.

3. Fragment according to claim 2, characterized in that it consists of a nucleotide sequence chosen from the sequences SEQ ID NO: 1, SEQ ID NO: 3 to SEQ ID NO: 18, SEQ ID NO: 20, SEQ ID NO: 22 to SEQ ID NO: 54, and their complementary sequences.

4. Single stranded nucleotide fragment of DNA, characterized in that it is obtained by reverse transcription of a nucleotide fragment according to any one of claims 1 to 3, or its complementary fragment.

5. Single-stranded nucleotide fragment of genomic DNA, characterized in that its transcription product is a nucleotide fragment according to any one of claims 1 to 3, or its complementary fragment.

6. Probe for the specific detection of bacteria of the genus Chlamydia, characterized in that it comprises a sequence of at least 12, preferably 18, or better still 20, contiguous nucleotide units, included in a sequence chosen from the sequences SEQ ID NO: 1 and SEQ ID NO: 3 to SEQ ID NO: 12, their complementary sequences and their equivalent sequences.

7. Probe according to claim 6, characterized in that it consists of a sequence of 12, preferably 18, or better still 20, contiguous nucleotide units, included in a sequence chosen from the sequences SEQ ID NO: 1 and SEQ ID NO: 3 to SEQ ID NO: 12 and their complementary sequences.

8. Probe for the specific detection of bacteria of the species Chlamydia trachomatis, characterized in that it comprises a sequence of at least 12, preferably 18, or better still 20, contiguous nucleotide units included in a sequence chosen from the sequences SEQ ID NO: 13 to SEQ ID NO: 18, SEQ ID NO: 20, and SEQ ID NO: 22 to SEQ ID NO: 24, their complementary sequences and their equivalent sequences.

9. Probe according to claim 8, characterized in that it consists of a sequence of 12, preferably 18, or better still 20, contiguous nucleotide units, included in a sequence chosen from the sequences SEQ ID NO: 13 to SEQ ID NO: 18, SEQ ID NO: 20, and SEQ ID NO: 22 to SEQ ID NO: 24 and their complementary sequences.

10. Probe for the specific detection of bacteria of the species Chlamydia pneumoniae, characterized in that it comprises a sequence of at least 12, preferably 18, or better still 20, contiguous nucleotide units, included in a sequence chosen from the sequences SEQ ID NO: 25 to SEQ ID NO: 39, their complementary sequences and their equivalent sequences.

11. Probe according to claim 10, characterized in that it consists of a sequence of 12, preferably 18, or better still 20, contiguous nucleotide units, included in a sequence chosen from the sequences SEQ ID NO: 25 to SEQ ID NO: 39 and their complementary sequences.

12. Probe for the specific detection of bacteria of the species Chlamydia psittaci, characterized in that it comprises a sequence of at least 12, preferably 18, or better still 20, contiguous nucleotide units, included in a sequence chosen from the sequences SEQ ID NO: 40 to SEQ ID NO: 54, their complementary sequences and their equivalent sequences.

13. Probe according to claim 12, characterized in that it consists of a sequence of 12, preferably 18, or better still 20, contiguous nucleotide units, included in a sequence chosen from the sequences SEQ ID NO: 40 to SEQ ID NO: 54 and their complementary sequences.

14. Probe according to any one of claims 6 to 13, characterized in that it is immobilized on a solid support.

15. Probe according to any one of claims 6 to 14, characterized in that it is labeled with a tracer agent, especially chosen from the radioactive isotopes, enzymes, in particular enzymes capable of acting on a chromogenic, fluorigenic or luminescent substrate such as a peroxidase or an alkaline phosphatase, chromophoric chemical compounds, chromogenic, fluorigenic or luminescent compounds, analogs of nucleotide bases, and ligands such as biotin.

16. Therapeutic probe for the treatment of infections due to a determined species of Chlamydia, characterized in that it comprises a sequence of at least 12, preferably 18, or better still 20, contiguous nucleotide units, included in a sequence chosen from the sequences SEQ ID NO: 1, SEQ ID NO: 3 to SEQ ID NO: 18, SEQ ID NO: 20, SEQ ID NO: 22 to SEQ ID NO: 54, their complementary sequences and their equivalent sequences.

17. Primer for the reverse transcription of a 23S ribosomal RNA sequence of a bacterium of the genus Chlamydia, characterized in that it comprises a sequence of at least 12, preferably 18, or better still 20, contiguous nucleotide units, included in a nucleotide sequence chosen from the sequences SEQ ID NO: 1, SEQ ID NO: 3 to SEQ ID NO: 18, SEQ ID NO: 20, SEQ ID NO: 22 to SEQ ID NO: 54, their complementary sequences and their equivalent sequences.

18. Primer for the enzymatic amplification of at least one nucleic acid sequence, such as amplification by chain polymerization reaction, characterized in that it comprises a sequence of at least 12, preferably 18, or better still 20, contiguous nucleotide units, included in a nucleotide sequence chosen from the sequences SEQ ID NO: 1, SEQ ID NO: 3 to SEQ ID NO: 18, SEQ ID NO: 20, SEQ ID NO: 22 to SEQ ID NO: 54, their complementary sequences and their equivalent sequences.

19. Reagent for detecting and/or identifying and/or quantifying at least one species of Chlamydia, characterized in that it comprises at least one probe according to any one of claims 6 to 15.

20. Reagent according to claim 19, characterized in that it comprises a capture probe according to claim 14, and a detection probe according to claim 15.

21. Reagent according to claim 19, characterized in that it additionally comprises a primer according to claim 17 and/or a primer according to claim 18.

22. Procedure for detecting and/or identifying and/or quantifying at least one species of Chlamydia, in a biological sample, capable of containing at least one nucleic acid of said species, characterized in that it comprises the steps consisting in contacting said sample with at least one probe according to any one of claims 6 to 15, and in detecting the possible formation of a hybridization complex between said probe and said nucleic acid.

23. Process according to claim 22, characterized in that said sample is contacted with a first probe according to claim 6 or 7, and possibly 14 or 15, and a second probe according to claim 8 to 13, and possibly 14 or 15.


SEQUENCE LISTING (60 sequences)

SEQ ID NO: 1 - RNA, 22 nt, Chlamydia sp.
gauacagggugauagucccgua

SEQ ID NO: 2 - RNA, 7 nt, Chlamydia sp.
uaguccc

SEQ ID NO: 3 - RNA, 29 nt, Chlamydia sp.
cuagucugaaucuggggagaccacucucc

SEQ ID NO: 4 - RNA, 23 nt, Chlamydia sp.
guuaagcacgcggacgauuggaa

SEQ ID NO: 5 - RNA, 74 nt, Chlamydia sp.
cacuaugcaaaccucuaaggggaaguauauggugugacgccugcccaaugccaaaagguuaaagggauauguca

SEQ ID NO: 6 - RNA, 30 nt, Chlamydia sp.
uggugaauggccgccguaacuauaacggug

SEQ ID NO: 7 - RNA, 28 nt, Chlamydia sp.
auagugaaccaguacugugaaggaaagg

SEQ ID NO: 8 - RNA, 25 nt, Chlamydia sp.
uugcaugaugagccagggaguuaag

SEQ ID NO: 9 - RNA, 13 nt, Chlamydia sp.
cuauuuaugacca

SEQ ID NO: 10 - RNA, 25 nt, Chlamydia sp.
ucuuguucucuccgaaauaacuuua

SEQ ID NO: 11 - RNA, 33 nt, Chlamydia sp.
cugaauucuagcgggggccuaccggcuuaccaa

SEQ ID NO: 12 - RNA, 60 nt, Chlamydia sp.
gaguccgggagauagacagcgggggcuaagcuucguugucgagaggggaacagcccagac

SEQ ID NO: 13 - RNA, 73 nt, Chlamydia trachomatis
agacaguuggaauguuggcuuagaggcagcaaucauuuaaagagugcguaacagcucaccaaucgagaaucau

SEQ ID NO: 14 - RNA, 31 nt, Chlamydia trachomatis
cuccuaguugaacacaucuggaaagauggau

SEQ ID NO: 15 - RNA, 29 nt, Chlamydia trachomatis
gacgaaaggagagaaagaccgaccucaac

SEQ ID NO: 16 - RNA, 20 nt, Chlamydia trachomatis
caauggcccguaagggucaa

SEQ ID NO: 17 - RNA, 41 nt, Chlamydia trachomatis
gcuaaacggcgagguuaagggauauacauuccggagccgga

SEQ ID NO: 18 - RNA, 16 nt, Chlamydia trachomatis
aagagucguuugguuu

SEQ ID NO: 19 - RNA, 11 nt, Chlamydia trachomatis
cuaguaccugu

SEQ ID NO: 20 - RNA, 18 nt, Chlamydia trachomatis
gaugauucgaagacaguu

SEQ ID NO: 21 - RNA, 11 nt, Chlamydia trachomatis
gucgauaagac

SEQ ID NO: 22 - RNA, 16 nt, Chlamydia trachomatis
agaguccguagagcga

SEQ ID NO: 23 - RNA, 19 nt, Chlamydia trachomatis
ggucgcgaucaaggggaau

SEQ ID NO: 24 - RNA, 31 nt, Chlamydia trachomatis
uuuuagggugacuauggaacgauaggagccc

SEQ ID NO: 25 - RNA, 26 nt, Chlamydia pneumoniae
cgauaacaugggaucuuaaguuuuag

SEQ ID NO: 26 - RNA, 22 nt, Chlamydia pneumoniae
ucuggaaaguugaacgauacag

SEQ ID NO: 27 - RNA, 36 nt, Chlamydia pneumoniae
ucccguaaacgaaaaaacaaaagacgcuaaucgaua

SEQ ID NO: 28 - RNA, 37 nt, Chlamydia pneumoniae
ucggagaccuauaacucuucggaguaaugguugacgg

SEQ ID NO: 29 - RNA, 61 nt, Chlamydia pneumoniae
ggaguuaaguuaaacggcgagauuaagggauuuacauuccggagucgaagcgaaagcgagu

SEQ ID NO: 30 - RNA, 32 nt, Chlamydia pneumoniae
ucaucgcgccaauaaugaucgggcucaagcau

SEQ ID NO: 31 - RNA, 19 nt, Chlamydia pneumoniae
cggguguauauuauguaua

SEQ ID NO: 32 - RNA, 23 nt, Chlamydia pneumoniae
cacaaugagacugguuaguaggc

SEQ ID NO: 33 - RNA, 37 nt, Chlamydia pneumoniae
gccucuuaaggugauuacccagcgguaugagccucgg

SEQ ID NO: 34 - RNA, 37 nt, Chlamydia pneumoniae
uauucagcagugaagguauaccgaaaggagugcugga

SEQ ID NO: 35 - RNA, 47 nt, Chlamydia pneumoniae
aaguaacgauaaaggaagugaaaaucuuccucgccguaagcccaagg

SEQ ID NO: 36 - RNA, 26 nt, Chlamydia pneumoniae
agcguuuuagucguuugauuuagaca

SEQ ID NO: 37 - RNA, 65 nt, Chlamydia pneumoniae
gguugcagcauugguaagacuuuguggaggaccgaaccaguacauguugaaaaauguuuggauga

SEQ ID NO: 38 - RNA, 31 nt, Chlamydia pneumoniae
uuagccucggauauuaaguuuuuggggguag

SEQ ID NO: 39 - RNA, 44 nt, Chlamydia pneumoniae
uuaugcuaagugaguaaggaagugauaauucuaagacaguugga

SEQ ID NO: 40 - RNA, 26 nt, Chlamydia psittaci
cgauaacaugggcucuuaaguuuuag

SEQ ID NO: 41 - RNA, 22 nt, Chlamydia psittaci
ucuggaaaguagaacgauacag

SEQ ID NO: 42 - RNA, 36 nt, Chlamydia psittaci
ucccguagacgaaaaaacaagagacucuauucgaua

SEQ ID NO: 43 - RNA, 39 nt, Chlamydia psittaci
ucggagaccuauaacuucuuaggaagucaugguugacgg

SEQ ID NO: 44 - RNA, 61 nt, Chlamydia psittaci
ggaguuaaucuaaacggcgagguuaagggaucuacauuccggagccgaagcgaaagcgagu

SEQ ID NO: 45 - RNA, 32 nt, Chlamydia psittaci
ucauugcgccaauaauaaucgggcucaagcau

SEQ ID NO: 46 - RNA, 19 nt, Chlamydia psittaci
cggguguauauuauauaua

SEQ ID NO: 47 - RNA, 23 nt, Chlamydia psittaci
cacaaugagaccgguuaguaggc

SEQ ID NO: 48 - RNA, 38 nt, Chlamydia psittaci
gccucuuagggugauugccuuuacggcaugagcuccgg

SEQ ID NO: 49 - RNA, 37 nt, Chlamydia psittaci
uauucagcagugaagguauaccguaaggagugcugga

SEQ ID NO: 50 - RNA, 47 nt, Chlamydia psittaci
aaguaacgauaaaggaagugaaaaucuuccucgccguaagcacaagg

SEQ ID NO: 51 - RNA, 25 nt, Chlamydia psittaci
  misc_feature (2)..(2): n = a or u or c or g or unknown or other
ancguuuagucguuugauuuagaca

SEQ ID NO: 52 - RNA, 65 nt, Chlamydia psittaci
  misc_feature (5)..(6): n = a or u or c or g or unknown or other
gguunnagcauggguaagacccugugaaggaccgaaccaguacauguugaaaaauguuuggauga

SEQ ID NO: 53 - RNA, 31 nt, Chlamydia psittaci
uuagccucggauauuaagcuuuuggggguag

SEQ ID NO: 54 - RNA, 44 nt, Chlamydia psittaci
uuaugcuaagugaguaaggaagugaugauucuaagacaguugga

SEQ ID NO: 55 - RNA, 2923 nt, Chlamydia trachomatis A
  misc_feature (1796)..(1796): n = a or u or c or g or unknown or other
aauuacagaccaaguuaauaagagcuauugguggaugccuuggcauugacaggcgaagaaggacgcgaauaccugcgaaaagcuccggcgagcuggugauaagcaaagacccggagguauccgaauggggaaacccgguagaguaauagacuaccauugcaugcugaauacauagguaugcaaagcaacaccugccgaacugaaacaucuuaguaagcagaggaaaagaaaucgaagagauucccuguguagcggcgagcgaaaggggaauagccuaaaccgagcugauaaggcucgggguuguaggauugaggauaaaggaucaggacuccuaguugaacacaucuggaaagauggaugauacagggugauagucccguagacgaaaggagagaaagaccgaccucaacaccugaguaggacuagacacgugaaaccuagucugaaucuggggagaccacucuccaaggcuaaauacuagucaaugaccgauagugaaccaguacugugaaggaaaggcgaaaagaccccuuguuaagggagugaaauagaaccugaaaccaguagcuuacaagcggucggagaccaauggcccguaagggucaagguugacggcgugccuuuugcaugaugagccagggaguuaagcuaaacggcgagguuaagggauauacauuccggagccggagcgaaagcgaguuuuaaaagagcgaagagucguuugguuuagacacgaaaccaagugagcuauuuaugaccagguugaagcauggguaaacuauguggaggaccgaacuaguaccuguugaaaaagguuuggaugaguugugaauaggggugaaaggccaaucaaacuuggagauaucuuguucucuccgaaauaacuuuaggguuagccucggauaauaagcuuuuggggguagagcacugaauucuagcgggggccuaccggcuuaccaacggaaaucaaacuccgaauaccagaagcgaguccgggagauagacagcgggggcuaagcuucguugucgagaggggaacagcccagaccgccgauuaaggucccuaauuuuaugcuaaguggguaaggaagugaugauucgaagacaguuggaauguuggcuuagaggcagcaaucauuuaaagagugcguaacagcucaccaaucgagaaucauugcgccgauaauaaacgggacuaagcauaaaaccgacaucgcgggugugucgauaagacacgcgguaggagagcguaguauucagcagagaagguguaccggaaggagcgcuggagcggauacuagugaagauccauggcauaaguaacgauaaagggagugaaaaucucccucgccguaagcccaagguuuccagggucaagcucgucuucccuggguuagucggccccuaaguugaggcguaacugcguagacgauggagcagcagguuaaauauuccugcaccaccuaaaacuauagcgaaggaaugacggaguaaguuaagcacgcggacgauuggaagaguccguagagcgaugagaacgguuaguaggcaaauccgcuaacauaagaucgggucgcgaucaaggggaaucuucgggggaaccgaugguguggagcgaggcuuucaagaaauaauuucuagcuguugauggugaccguaccaaaaccgacacaggugggcgagaugaauauucuaaggcgcgcgagauaacuuuuguuaaggaacucggcaaauuauccccguaacuucggaauaaggggagccuuuuagggugacuauggaacgauaggagcccnggggggccgcagagaaauggcccaggcgacuguuuagcaaaaacacagcacuaugcaaaccucuaaggggaaguauauggugugacgccugcccaaugccaaaagguuaaagggauaugucagcugcaaagugaagcauugaaccuaagcccuggugaauggccgccguaacuauaacggugcuaagguagcgaaauuccuugucggguaaguuccgaccugcacgaaugguguaacgaucugggcacugucucaacgaaagacucggugaaauuguaguagcagugaagaugcuguuuacccgcgaaaggacgaaaagaccccgugaaccuuuacuguacuuugguauugauuuuugguuuguuauguguaggauagccaggagacuaagaacacucuucuucaggagagugggagucaacguugaaauacuggucuuaacaagcugggaaucuaacauuauuccaugaaucuggaagauggacauugccagacgggcaguuuuacuggggcgguauccuccuaaaaaguaacggaggagcccaaagcuuauuucaucgugguuggcaaucacgaguagagcguaaagguauaagauagguugacugcaagaccaacaagucgagcagagacgaaagucgggcuuagugauccggcgguggaaaguggaaucgccgucgcuuaacggauaaaagguacuccggggauaacaggcugaucgccaccaagaguucauaucgacguggcgguuuggcaccucgaugucggcucaucgcauccuggggcuggagaaggucccaaggguuuggcuguucgccaauuaaagcgguacgcgagcuggguucaaaacgucgugagacaguuuggucucuauccuucgugggcgcaggauacuugagaggagcuguuccuaguacgagaggaccggaauggacgaaccaauggugugucgguuguuuugccaagggcauagccgaguagcuacguucggaaaggauaagcauugaaagcaucuaaaugccaagccucccucaagauaagguaucccaaugagacuccauguagacuacgugguugaaagguuggagguguaagcacaguaauguguucagcuaaccaauacuaau

SEQ ID NO: 56 - RNA, 30 nt, Chlamydia trachomatis
gaggucggucuuucucuccuuucgucuacg

SEQ ID NO: 57 - RNA, 35 nt, Chlamydia trachomatis
ccggggcuccuaucguuccauagucacccuaaaag

SEQ ID NO: 58 - RNA, 27 nt, Chlamydia trachomatis
cggucuuucucuccuuucgucuacggg

SEQ ID NO: 59 - RNA, 20 nt, Chlamydia pneumoniae
cgcuggguaaucaccuuaag

SEQ ID NO: 60 - DNA, 2924 bp, Chlamydia pneumoniae
aatttacagaccaagttgttaagagctattggcggatgccttggcattgacaggcgatgaaggatgcgtttacctgcagtaatcttcggtgagctggtatagagctatgacccggaggtatccgaatggggcaacccgatagactaatagtctatcattatatgttgaatacataggcatataaggcgacacccgctgaactgaaacatcttagtaagcggaggaaaagaaatcaaagagattccctgtgtagcggcgagcgaaaggggaacagcctaaaccatatttttaatatggggttgtagggtcgataacatgggatcttaagttttagttgaatacttctggaaagttgaacgatacagggtgatagtcccgtaaacgaaaaaacaaaagacgctaatcgatacctgagtagggctagacacgtgaaacctagtctgaatctggggagaccactctccaaggctaaatactagtcaatgacctatagtgaaccagtactgtgaaggaaaggtgaaaagaacccttgttaagggagtgaaatagaacctgaaaccagtagcttataagcggtcggagacctataactcttcggagtaatggttgacggcgtgccttttgcatgatgagccagggagttaagttaaacggcgagattaagggatttacattccggagtcgaagcgaaagcgagttttaaaagagcgttttagtcgtttgatttagacacgaaaccaagtgagctatttatgaccaggttgaagcattggtaagactttgtggaggaccgaaccagtacatgttgaaaaatgtttggatgagttgtgaataggggtgaaaggccaatcaaacttggagatatcttgttctctccgaaataactttagggttagcctcggatattaagtttttgggggtagagcactgaattctagcgggggcctaccggcttaccaacggaaatcaaactccgaataccaaaagcgagtccgggagatagacagcgggggctaagcttcgttgtcgagaggggaacagcccagaccgccgattaaggtccctaattttatgctaagtgagtaaggaagtgataattctaagacagttggaatgttggcttagaggcagcaatcatttaaagagtgcgtaacagctcaccaatcgagaatcatcgcgccaataatgatcggggctcaagcataaaaccgacatcgcgggtgtatattatgtatacgcggtaggagagtgtagtattcagcagtgaaggtataccgaaaggagtgctggagcggatactagtaaagatccatggcataagtaacgataaaggaagtgaaaatcttcctcgccgtaagcccaaggtttccagggtcaagctcgtcttccctgggttagtcggcccctaagtcgaggcacaaatgcgtagacgatggagcaacaggttaaatattcctgtaccacctaaaactttagcaatggaatgacggagtacgttaagcacgcggacgattggaaatgtccgtatcacaatgagactggttagtaggcaaatccgctaacacaaggtcgggttgtggttaagggaaatcttcggaggaactgatagtgtggcgcaaggctttcaagagataatttctagctgttgatggtgaccgtaccaagaccgacacaggtgggcgagatgagtattctaaggcgcgcgagataactttcgttaaggaactcggcaaattatccccgtaacttcggaataaggggagcctcttaaggtgattacccagcggtatgagcctcggggggccgcagagaaatggcccaggcgactgtttaacaaaaacacagcactatgcaaacctctaaggggaagtatatggtgtgacgcctgcccaatgccaaaaggttaaagggatatgtcagccgcaaggcaaagcattgaacccaagccctggtgaatggccgccgtaactataacggtgctaaggtagcgaaattccttgtcgggtaagttccgacctgcacgaatggtgtaacgatctgggcactgtctcaacgaaagactcggtgaaattgtagtagcagtgaagatgctgtttacccgcaaaaggacgaaaagaccccgtgaacctttactgtactttggtattgatttttgatttgttatgtgtaggatagccaggagactatgaacactcttcgttaggagggtgggagtcattgttgaaatactggtcttaacaagttgggagtctaacattactccatgaatctggagaatggacattgccagacgggcagttttactggggcggtatcctcctaaaaagtaacggaggagcccaaagcttatttcatcgtggttggcaatcacgagtagagcgtaaaggtataaaataggttgactgcaagacttacaagtcgagcagagacgaaagtcgggcttagtgatccggcggtggaaagtggaatcgccgtcgcttaacggataaaaggtactccggggataacaggctgatcgccaccaagagttcatatcgacgtggcggtttggcacctcgatgtcggctcatcgcatcctggggctggagaaggtcccaagggtttggctgttcgccaattaaagcggtacgcgagctgggttcaaaacgtcgtgagacagtttggtctctatcctttgtgggcgcaggatacttgaaaggagctgttcctagtacgagaggaccggaatggacgaaccaatggtgtgtcggttgttttgccaaaagcatagccgagtagctacgttcggaaaggataagcattgaaagcatctaaatgccaagcctcccttaagataaggtatccctatgagactccatgtagactacgtggttgataggttgggtgtgtacgcacagtaatgtgtttagctaaccaatactaat